(19) United States
(12) Patent Application Publication
(10) Pub. No.: US 2008/0018123 A1
(43) Pub. Date: Jan. 24, 2008

(54) Finger tip grip

(76) Inventor: Elizabeth Suzanne Cox, Baltimore, MD (US)

Correspondence Address: JOHN E. SIMMS JR., 8056 PHILADELPHIA ROAD, LOWER LEVEL, BALTIMORE, MD, 21237, US

(21) Appl. No.: 11/879,315

(22) Filed: Jul. 17, 2007

Related U.S. Application Data
(60) Provisional application No. 60/831,505, filed on Jul. 18, 2006.

(51) Int. Cl. A61F 13/10; A41D 13/08

(52) U.S. Cl. 294/25; 2/21

(57) ABSTRACT

A finger tip grip comprised of a generally oval membrane of natural rubber having a rough obverse surface for frictionally gripping a sheet of paper and a concave reverse surface for application on the ventral aspect of a human finger tip. The reverse surface is provided with adhesive for adhering the finger tip grip to human skin. The obverse surface is provided with projections to enhance the coefficient of friction. The membrane is sized to comfortably fit a finger tip and includes holes for ventilation. A removable protective film is affixed to the adhesive. The adhesive adheres the finger tip grip to a finger tip and provides advantageous friction for counting or reviewing papers and currency. The finger tip grip is disposable after use.

### REFERENCE TO RELATED APPLICATIONS

Applicant claims the benefit of Provisional Patent Application, Ser. No. 60/831,505, filed 18 Jul. 2006.

### BACKGROUND

Many tasks require individuals to leaf through numerous papers while making sure that each sheet is counted or observed. The smoothness of human skin contributes to the difficulty in separating papers page by page. Typically, individuals use a finger tip to rub a corner of a page with sufficient pressure to bind the page to the finger, in frictional engagement, so that a single page can be partially lifted to a point where it can be grasped. Various methods are employed to reduce the smoothness of the finger tip and thereby increase the friction imparted by a given amount of pressure. Friction may be increased by simply moistening the finger tip. Individuals, such as secretaries, bank tellers and cashiers, frequently maintain a damp sponge near their work station, and moisten their finger tips before counting a stack of papers, currency, receipts or other papers. Moisture quickly evaporates and becomes dispersed on the papers. A tacky balm has been developed to apply on finger tips, to increase the friction. The tacky balm is also dispersed on the papers as a greasy residue, and though it remains on the finger tips longer than water, continued re-application is still necessary. Also, both the tacky balm and the damp sponge may accumulate bacteria and become unsanitary.

As an alternative to coatings, for the finger tip, rubber sheaths, commonly known as “rubber fingers”, in the general shape of a sewing thimble, have been developed. The rubber sheaths fit loosely over the end of an individual's finger and provide a high friction surface for pushing a sheet of paper to separate it from an adjacent sheet. The rubber sheaths may be used continuously in work which requires repeatedly turning pages, for viewing, or separating currency or other papers for counting; however, the rubber sheaths tend to slip off during rapid movement. Since the rubber sheaths cover the entire end to the finger, perspiration inside the sheath is a common problem, which is unsanitary and increases the tendency of the sheath to slip off Also, the rubber sheaths are relatively thick and are too bulky to wear for other tasks, such as dialing a telephone, operating a calculator, typing or writing. Individuals having relatively long fingernails find that the rubber sheaths do not fit properly. Typically, individuals remove the rubber sheaths in order to do something other than separating currency or other papers and replace the rubber sheath to begin or continue a task requiring the separation of sheets of paper. The thickness of the rubber sheaths inhibits tactile sensitivity and reduces dexterity in handling a sheet of paper, once it is separated. The rubber sheaths also collect dust, dirt, and bacteria during repeated uses, which causes unsightly appearance and a possible health concern.

There is a need for a finger tip grip, having a friction coefficient comparable to a rubber sheath, but which is thinner, unobtrusive and much more comfortable to wear. There is a need for a finger tip grip which may be worn for a considerable length of time and which will not interfere with manual dexterity in performing a wide variety of tasks. There is a need for a finger tip grip which is disposable for avoiding the unsightly appearance and health concerns associated with the repeated use of a conventional rubber sheath, tacky balm, or wet sponge.

### SUMMARY OF THE INVENTION

The present invention is a finger tip grip comprising a curved membrane having a rough obverse surface and an opposite concave reverse surface. The membrane is formed in a generally oval shape having a central portion and a peripheral edge. The thickness of the membrane may be graduated with an area of relatively greater thickness proximate to the central portion, for increased durability, and tapering to a lesser thickness proximate to the peripheral edge. The membrane may also include a line of perforation, running from a point on the peripheral edge to the central portion, to facilitate tearing, for size adjustment. An adhesive is disposed on the reverse surface. The membrane is sized to partially cover the ventral aspect of a human finger tip.

The membrane is provided with a removable protective film affixed to the adhesive. A user may remove the protective film and apply the reverse surface to the ventral aspect of a finger tip, securing the membrane by the adhesive. Before the membrane is applied, the user may tear the sheet along the line of perforation to form adjacent flaps and overlay the flaps to a varying degree to accentuate the concave curvature of the reverse surface and improve the fit. The membrane may be provided with a plurality of holes to allow ventilation of the skin on the user's finger tip. The obverse surface is provided with a plurality of projections or abrasions to enhance frictional engagement with a sheet of paper.

A user may apply one or more finger tip grips to one or more corresponding finger tips and sort papers page by page, or separate bills or currency, using the increased coefficient of friction of the finger tip grips to aid in sliding each sheet to separate it from an adjacent sheet. Other tasks may be readily performed without removing the finger tip grips and without hindering normal dexterity. At the end of a period of use, the finger tip grips may be removed, by peeling, and may be discarded.

It is an object of the present invention to provide a finger tip grip having a rough obverse surface capable of frictionally engaging a paper.

It is another object of the present invention to provide a finger tip grip which will not interfere with normal manual dexterity.

It is a further object of the present invention to provide a finger tip grip which is disposable following use to avoid the accumulation of dirt and bacteria associated with repeated use.

### BRIEF DESCRIPTION OF THE DRAWINGS

The present invention may be more fully understood with reference to the drawing figures in which:

FIG. 1 is a side perspective view of the finger tip grip of the present invention.

FIG. 2 is a top plan view of the finger tip grip of the present invention.

FIG. 3 is a side perspective view of the finger tip grip of the present invention with the protective film partially removed.

FIG. 4 is a perspective view of the finger tip grip of the present invention, and in the alternative embodiment, applied on a human finger tip.

FIG. 5 is a bottom plan view of the finger tip grip of the present invention showing a flap formed by tearing along the line of perforation.

### DETAILED DESCRIPTION OF THE PREFERRED EMBODIMENT

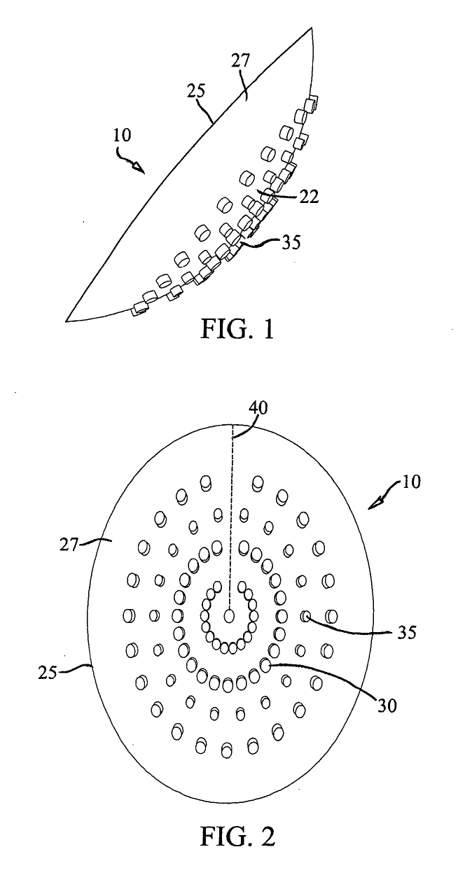

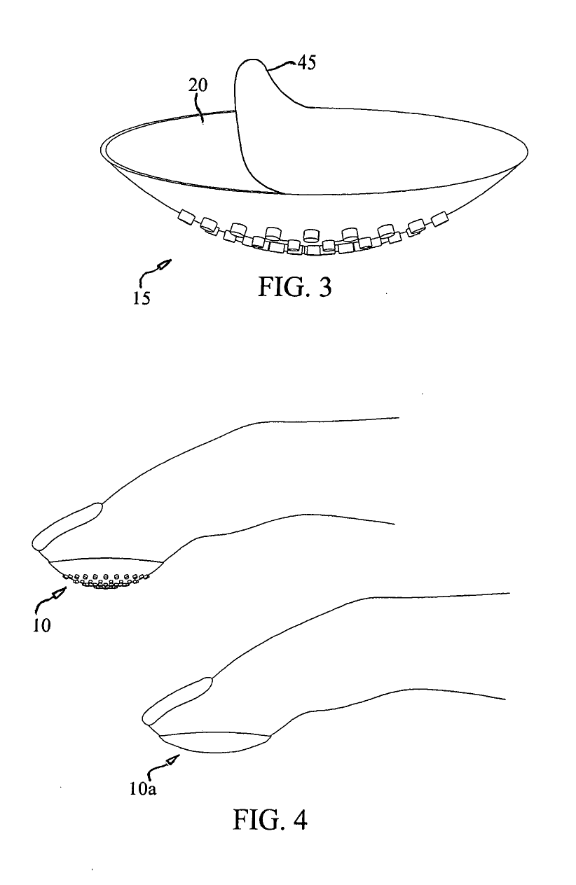

The finger tip grip, of the present invention, as shown in FIGS. 1-5 is a conveniently attachable and disposable membrane 10, designed to be applied to the ventral aspect of a human finger tip to assist in easily separating sheets of paper, or bills, one by one. The membrane 10 includes a rough obverse surface 15 and an opposite concave reverse surface 20, as shown in FIG. 3. Adhesive is disposed on the reverse surface 20. The membrane 10 is formed of flexible material in a generally oval shape, with a central portion 22 and a peripheral edge 25. Natural rubber, molded to preselected dimensions using conventional techniques, is the preferred material for forming the membrane 10. Natural rubber is sufficiently flexible to conform to the shape of a finger tip and is sufficiently rough to frictionally engage a sheet of paper. Alternatively, latex rubber or fiber mesh coated with natural rubber or latex rubber are suitable materials for forming the membrane. It is intended that the membrane 10 be sized to partially cover the ventral aspect of a human finger tip. It is preferred that the thickness be graduated from relatively greater thickness at the central portion 22 and tapering to lesser thickness proximate to the peripheral edge 25, for best fit. The thicker central portion 22 provides improved durability. For certain tasks, an alternative embodiment membrane 10a similar to the membrane 10 and having uniform thickness throughout is provided, as shown in FIG. 4.

It is intended that the finger tip grips would be made available in a range of sizes to fit the finger tips of individuals of varying size. It is preferred that the dimensions of the oval membrane 10 and alternative embodiment membrane 10a range from small size of approximately 2.2 cm.×1.6 cm to extra large size of approximately 2.9 cm×2.4 cm., with intermediate sizes within the range. The thickness of the membrane 10 ranges from approximately 0.004 cm. to 0.008 cm. at the central portion 22 and from 0.003 cm. to 0.004 cm. proximate to the peripheral edge 25. It is preferred that the area proximate to the peripheral edge 25, is formed as an integral circumferential band 27 having a width of approximately 0.3 cm. to 0.4 cm. and a uniform thickness within the range given for the area proximate to the peripheral edge 25, as shown in FIGS. 1 and 2. It is intended that a band 27 having a width generally at the lower end of the given range is suitable for smaller sized finger tip grips and a band 27 having greater width is suitable for larger sized finger tip grips. The thickness of the central portion 22 is selected to maximize durability without undue bulk and the lesser thickness of the band 27 and peripheral edge 25 is selected to minimize the discontinuity between the obverse surface 15 and the skin of the individual, without making the membrane 10 unduly fragile. The alternative embodiment membrane 10a, having uniform thickness throughout, is preferably formed with a thickness of approximately 0.003 cm. to 0.004 cm. Durability is reduced but tactile sensitivity is enhanced, making the alternative embodiment membrane 10a ideal for certain particular tasks.

It is preferred that the membrane 10 be provided with a plurality of holes 30, as shown in FIG. 2, in the central portion 22, for ventilation. The holes 30 are preferably approximately 0.005 cm. diameter. The alternative embodiment membrane 10a is preferably formed without ventilation holes 30, when the thickness is in the range given for the band 27, as the alternative embodiment membrane 10a becomes too fragile. It is also preferred that the obverse surface 15 of the membrane 10 be provided with a plurality of projections 35, for enhancing the coefficient of friction, as shown in FIGS. 1 and 2. The projections 35 may be formed of the same material as the membrane 10 and may preferably be formed integrally with the membrane 10. The projections 35 are preferably approximately 0.005 cm. long and 0.005 cm. in circumference. The alternative embodiment membrane 10a is preferably formed without projections 35. The roughness of the obverse surface of the alternative embodiment membrane 10a may be enhanced by abrading the surface.

In order to improve the fit of the finger tip grip, as more particularly described below, a perforation line 40 is provided from the peripheral edge 25 to the central portion 22, to facilitate tearing, as shown in FIG. 2. It should be noted that, in the alternative embodiment membrane 10a, the elasticity allows for conformity with the curvature of a user's finger tip and the perforation line 40 is generally unnecessary. A removable protective film 45 is affixed to the adhesive on the reverse surface of the membrane 10, to prevent premature attachment of the adhesive and to prevent the accumulation of foreign matter on the adhesive, as shown in FIG. 3. It is intended that the reverse surface of the alternative embodiment membrane 10a be coated with adhesive and provided with protective film, as well. It is preferred that the adhesive be disposed over the entire reverse surface 20 and that the adhesive be of conventional variety typically used for waterproof plastic bandages. The protective film 45 is preferably of conventional design.

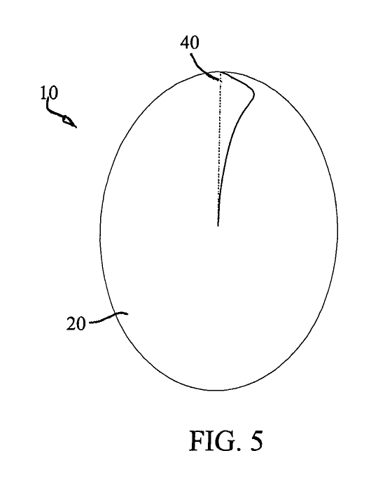

In use, an individual may select a finger tip grip of appropriate size and peel the protective film 45 from the reverse surface 20, to expose the adhesive. The reverse surface 20 may be applied to the ventral aspect of a finger tip and the membrane 10 or alterative embodiment membrane 10a may be pressed to cause the adhesive to adhere to the skin of the finger tip, as shown in FIG. 4. If desired, before applying the finger tip grip, the membrane 10 may be torn along the perforation line 40, to form adjacent flaps, as shown in FIG. 5. The flaps may be overlayed, one upon the other, to varying degrees, to deepen the concave curvature of the reverse surface 20, and more closely match the curvature of the finger tip.

With the finger tip grip affixed to the finger tip of the individual, a normal motion of handling papers by pressing the finger tip on a corner of a sheet and sliding the sheet to separate the sheet from a next sheet may be used. The separated sheet may be lifted to be counted or turned, as necessary. Pages bound in a volume, packs of currency, or any stack of papers may be conveniently separated one by one. The rough texture of the rubber, on the obverse surface 15 together with the projections 35 provide an advantageous coefficient of friction between the finger tip grip and the page of paper. The holes 30 provide ventilation for the skin, in the area of the finger tip grip. The finger tip grip may be comfortably worn for an extended period of time. Tasks requiring repeated counting or paging through multiple papers in a stack or in a multi-page document may be efficiently performed. Also, the individual may alternate with other tasks, such as using a telephone, calculator, keyboard, writing instrument, or other tasks requiring manual dexterity, without removing the finger tip grip and without interference from the finger tip grip. At the end of a period of use, such as at the end of a work day, the finger tip grip may be peeled off the finger tip and discarded.

It will be appreciated by those skilled in the art that the finger tip grips will be useful in providing an advantageous coefficient of friction for a wide variety of tasks, where a reliable grip, for handling an object, is required and the smoothness of human skin limits the effectiveness of the hand grip. In addition to the handling of currency and other paper, the handling of material such as glass and other relatively slippery items may be performed more efficiently with the use of finger tip grips. Also, athletes who catch balls or grip sports equipment may benefit from the use of finger tip grips. An individual may apply a plurality of finger tip grips to a plurality of fingers in one to one correspondence, if a task so requires. Having fully described the present invention, it may be understood that minor variations may be introduced without departing from the scope of the invention as disclosed and claimed herein.

I claim:

1. A finger tip grip comprising:
a membrane having a rough obverse surface and an opposite reverse surface;
said reverse surface having a concave curvature and having adhesive disposed thereon;
said membrane being sized to partially cover the ventral aspect of a human finger tip;
whereby said reverse surface may be applied to a human finger tip and retained by said adhesive for facilitating the handling of papers or objects.

2. The finger tip grip of claim 1, wherein said membrane is generally oval.

3. The finger tip grip of claim 1, wherein said membrane is provided with a plurality of holes, for ventilation.

4. The finger tip grip of claim 1, wherein said obverse surface is provided with a plurality of projections, for promoting frictional engagement.

5. The finger tip grip of claim 1, further including a removable protective film affixed to said adhesive.

6. The finger tip grip of claim 1, wherein said membrane is provided with a line of perforation to facilitate tearing, for size adjustment.

7. The finger tip grip of claim 1, wherein said membrane is of graduated thickness having a central portion of relatively greater thickness tapering to a circumferential band having relatively lesser thickness.

8. The finger tip grip of claim 7 wherein:
the thickness of said central portion measures in the range of approximately 0.004 to 0.008 centimeters; and
the thickness of said circumferential band measures in the range of approximately 0.003 to 0.004 centimeters.

9. The finger tip grip of claim 1, wherein said membrane is of uniform thickness measuring in the range of approximately 0.003 to 0.004 centimeters.

10. The finger tip grip of claim 1, wherein said membrane is formed of natural rubber.

11. The finger tip grip of claim 1, wherein said membrane is formed of latex rubber.

12. The finger tip grip of claim 1, wherein said membrane is formed of fiber mesh coated with rubber.
